(19) United States
(12) Patent Application Publication
(10) Pub. No.: US 2019/0015458 A1
(43) Pub. Date: Jan. 17, 2019

(54) A Composition, A Treatment Method and An Application Thereof

(71) Applicant: Blue Elegant Biotech Co., Ltd., Guangdong (CN)

(72) Inventors: Xiaoping Chen, Guangdong (CN); Li Qin, Guangdong (CN)

(21) Appl. No.: 15/748,092

(22) PCT Filed: Oct. 13, 2017
(86) PCT No.: PCT/CN2017/106077

§ 371 Date: Jan. 26, 2018

(30) Foreign Application Priority Data
Jan. 20, 2017 (CN) 201710041702.8

(51) Int. Cl. A61K 35/68; A61P 35/04

(52) U.S. Cl. CPC A61K 35/68; A61P 35/04

(57) ABSTRACT

The present invention relates to the field of treatment of tumor, and especially to a composition comprising a plasmodium, a treatment method and an application thereof. The composition of the present invention has therapeutic effects on colorectal carcinoma, lung carcinoma, breast carcinoma, gastric carcinoma and hepatic carcinoma etc., can inhibit the growth of tumor and prolong the life of the tumor patients, whereas has no therapeutic effect on melanoma and lymphoma; meanwhile, the present invention describes that the long-term plasmodium infection has better therapeutic effect on tumors, and the plasmodium immunotherapy of the present invention does not take the fever time as a course standard when treating tumors, but should be used to extend the duration of plasmodium infection as much as possible until the progression of tumors can be controlled under the premise of protecting the organ functions and life safety of the patients.

### TECHNICAL FIELD

The present invention relates to the field of treatment of tumor, and especially to a composition, a treatment method and an application thereof.

### BACKGROUND ART

Tumor is currently a class of disease that seriously threatens human health and life worldwide, it is estimated that there are 14 million new cases each year around the world by World Health Organization (WHO) in 2012. There are about 4.292 million new cases of tumor and 2.814 million cases of death in 2015 in China, which is equivalent to an average of 12,000 new cases and 7,500 cases of death every day, respectively. At present, the clinical treatment of a tumor is mainly focused on the comprehensive treatment, but it has poor efficacy on advanced solid tumors.

Malaria is widely prevalent all around the world, it is an insect-borne infectious disease which is seriously harmful to human health, and has been classified as one of the three major infectious diseases including AIDS and Tuberculosis by WHO in the world. The incidence of malaria in the world is mainly concentrated in tropical African countries, accounting for about 90% of the total incidence of malaria. There are mainly five types of plasmodia that infect humans: Plasmodium (P.) falciparum, P. vivax, P. malariae, P. ovale and P. knowlesi. The most common is P. falciparum and P. vivax, while P. falciparum infection is the most deadly malaria infection. The life history of a plasmodium can be divided into two stages, i.e., the initial stage of asexual proliferation and sexual proliferation in the human body and the stage of sexual proliferation and sporogony in the anopheles body. There are mainly four types of plasmodia in mice: P. chabaudi, P. yoelii, P. berghei and P. venckei, wherein, P. chabaudi, P. yoelii, and P. berghei are more suitable for the study of malaria immunology, pathogenesis and malaria vaccines.

Some parasite infections such as Trypanosoma Cruze, Toxoplasma gondii, Toxocara canis, and Kashgar acanthamoeba can improve the survival rate of the mice with cancer. Reports using animal models suggest that there is a link between the plasmodium infection and the morbidity/mortality of certain cancers. It has been reported by Trager et al. that Rous I tumor can be inhibited by the plasma from chicken infected with malaria parasite [Trager, W. and R. B. McGhee. Inhibition of chicken tumor I by plasma from chickens infected with an avian malaria parasite. Proc Soc Exp Biol Med 1953, 83(2): 349-352]. It has been reported by Angsubhakorn et al. that aflatoxin-induced liver cancer in rat can be prevented by the plasmodium infection (Angsubhakorn S, Bhamarapravati N, Sahaphong S, Sathiropas P. Reducing effects of rodent malaria on hepatic carcinogenesis induced by dietary aflatoxin B1. Int J Cancer 1988; 41:69-73). A study group led by Professor Xiaoping Chen took murine Lewis lung carcinoma as a model (the animal model of NSCLC) to explore the effects of plasmodium infection on the growth and metastasis of tumor and related mechanisms thereof. At present, the experimental results from this research group have demonstrated that plasmodium infection can significantly inhibit the growth and metastasis of murine Lewis lung carcinoma, and prolong the survival time of tumor-bearing mice [Chen L, He Z, Qin L, Li Q, Shi X, et al. Antitumor Effect of Malaria Parasite Infection in a Murine Lewis lung carcinoma Model through Induction of Innate and Adaptive Immunity. PLoS ONE 2011, 6: e24407].

CN101480489A discloses a new use of plasmodium circumsporozoite protein in anti-tumor proliferation and metastasis, indicating that the circumsporozoite protein of human P. falciparum has a function in inhibiting tumor proliferation and metastasis at the cellular level.

Although it has been shown in the animal experiments that plasmodium infection can significantly inhibit the growth of tumor in mice and prolong the survival time of tumor-bearing mice, it is worth further studying that on which tumor it has better effect specifically, and how to establish an immune response.

### SUMMARY

In view of the currently existing technical problems, the present invention provides a composition, a treatment method and an application thereof. The composition has therapeutic effects on various solid tumors such as colorectal carcinoma, gastric carcinoma, lung carcinoma, breast carcinoma, and hepatic carcinoma, etc., which can inhibit tumor growth and prolong the life of patients, but has no therapeutic effect on two types of tumor namely melanoma and lymphoma.

To achieve this objective, the present invention adopts the following technical solutions:

In one aspect, the present invention provides a composition, which comprises a plasmodium.

In the present invention, the composition is used for the treatment of cancer, has therapeutic effects on various solid tumors such as colorectal carcinoma, gastric carcinoma, lung carcinoma, breast carcinoma, and hepatic carcinoma, etc., the plasmodium infection acts by inducing a strong anti-tumor immune response, including activation of innate immune cells such as dendritic cells (DCs), macrophages, NK cells, and secretion of cytokines such as IFN-γ, TNF-α, etc., as well as stimulating the body to produce an antigen-specific anti-tumor immune response.

According to the present invention, the plasmodium is any one or a mixture of at least two of P. falciparum, P. vivax, P. malariae, P. ovale or P. knowlesi, preferably P. vivax. There are mainly five types of plasmodia that infect humans: P. falciparum, P. vivax, P. malariae, P. ovale and P. knowlesi. The most common is P. falciparum and P. vivax, wherein the P. falciparum infection can cause severe anemia, and about 1% of the infected person may develop cerebral malaria which will endanger the lives of patients, while P. vivax infection rarely endangers the lives of patients, therefore it is preferable to use P. vivax for the treatment.

According to the present invention, the inoculation of plasmodium causes a long-term plasmodium infection, the longer the infectious course of plasmodium is, the more obvious the inhibitory effect on tumors is. The long-term plasmodium infection is a plasmodium infection lasting to a chronic phase, which will be maintained for a period of time, then an anti-malarial drug will be administrated to terminate the infection. Alternatively, the infection will not be terminated, so that the patients will be in a state of parasite-carrying, and will come into a chronic phase through about 6-8 weeks of acute infectious stage (the clinical manifestations of which are a typical chill, a fever, and a sweating, then disappearing of fever) after the plasmodium infection, at which time only a small amount of plasmodia can be detected in the peripheral blood, and there is no clinical symptom of acute stage.

According to the present invention, the number of plasmodia which can successfully infect the tumor patient is feasible, and varies with the individual differences in tumor patients. Those skilled in the art can adjust the number of plasmodia according to the actual needs. The inoculation amount of the plasmodium in the present invention is not less than 100 active plasmodium-infected red blood cells, or no less than 5 active plasmodium sporozoites.

According to the present invention, the composition is useful for the preparation of a medicament for the treatment of tumors.

Preferably, the composition further comprises a pharmaceutically acceptable adjuvant.

Preferably, the adjuvants is any one or a combination of at least two of a excipient, a diluent, a carrier, a flavoring agent, a binder and a filler.

In a second aspect, the present invention provides a method for preparing the composition as described in the first aspect, comprising the steps of: cryopreserving and resuscitating the plasmodia or the plasmodium sporozoites, then preparing them into the composition.

The specific steps for cryopreserving and resuscitating the parasites are as follows:

1) Cryopreserving the plasmodia:
(1) whole blood containing the plasmodia is centrifuged at 300 g (1200 rpm) for 10 min (with low brake), then the plasma is transferred to another 50 mL centrifuge tube;
(2) the layer of white blood cell is removed, 2-fold volume of 1640 culture medium is added, then mixed well, the mixture is centrifuged at 300 g for 5 minutes (with low brake);
(3) the 50 mL centrifuge tube is placed on a vortex shaker, and an equal volume of 28% glycerin cryopreservation solution (7-8 mL) is added dropwise while shaking, then incubated for 5 minutes at room temperature;
(4) aliquot into cryovials in batches of 1 ml per vial
(5) the frozen vials are put into a cryopreservation box, which is preserved in liquid nitrogen directly;

Quality control of cryopreservation: an appropriate amount of RBC-28% glycerin cryopreservation solution mixture before cryopreservation is taken and an appropriate amount of RPMI 1640 culture medium is added thereto, the mixture is cultured at 37° C. and incubated in a carbon dioxide incubator for 72h, then the color of the culture medium is observed to confirm no turbidity.

2) Resuscitating the plasmodia:
(1) advance preparations: a water bath kettle (37° C.), a 50 mL centrifuge tube, 3.5% NaCl, a sodium chloride injection, a centrifuge are prepared, and labels and lists, etc., are printed; (2) the labels of the frozen vials, etc. are checked;
(3) the cryopreserved blood containing the plasmodia is taken (1.0 mL per tube), and placed into a water bath at 37° C. for 1-3 minutes to be thawed;
(4) the thawed blood containing the plasmodia is transferred to a 15 mL centrifuge tube and an equal volume of 3.5% NaCl is slowly added along the wall of the tube, which is gently blown and beaten to mix well, then stands still at room temperature for 5 minutes, centrifuged at 300 g for 5 minutes, and the supernatant is discarded;
(5) 5-fold volume of 0.9% NaCl is slowly added along the wall of the tube, which is gently blown and beaten to mix well, centrifuged at 300 g for 5 minutes, and the supernatant is discarded;
(6) physiological saline is added until the hematocrit (HCT) reaches 50%, mixed well, the density of the red blood cell is counted and recorded, temporarily stored at 4° C. for clinical inoculation.

Quality control of resuscitation: after the inoculation, a blood smear of the remaining blood sample is prepared, and then observed by microscopy for the infection rate and the parasite status before injection. Observations are then recorded.

The specific steps for cryopreserving and resuscitating the plasmodium sporozoites are as follows:

### 1. Membrane Feeding Parasite to Anopheles Mosquitoes

(1) Previous preparations: Volunteers are monitored for parasitemia and gametocyte formation, and 2 ml of peripheral blood is collected via vein. The anopheles are starved 24 hours in advance (300 or more of anopheles mosquitoes);
(2) before feeding the anopheles with blood, the room temperature is adjusted to 26° C., and the membrane blood-feeding system is made ready. The collected peripheral blood is added to the membrane blood-feeding system, and the anopheles are allowed to feed for 30 minutes;
(3) the unfed mosquitoes are removed, and left mosquitoes are labeled, and placed into an incubator at 26° C., sugar water cotton containing 10% glucose+0.05% PABA is changed every day.

### 2. Oocyst Examination

(1) The oocysts are examined at day 7-10 after the infection by blood-feeding, and 10 mosquitoes are dissected to calculate the proportion of positive oocysts mosquitoes;
Notes: 1) Standard oocysts classification, +: 1-10, ++: 11-100, and +++: 101 or more;
(2) If the number of anopheles with “++” or more oocyst is <50%, 20 anopheles are reexamined.

### 3. Sporozoite Examination

(1) The sporozoites are examined at day 14-16 after the infection by feeding blood, 20 mosquitoes are dissected, and the total number of sporozoites and the average number of sporozoites per mosquitoes are calculated.

Precautions: 1) A cell counting chamber is used for counting, the number of all sporozoites in the big grid is counted, and followed the principle of “counting the upper rather than the lower, and counting the left rather than the right”; (2) an average value of three times of counting are taken to minimize the errors when counting.

### 4. Sporozoite Acquisition

(1) The mosquitoes are taken out at day 14-20 after the infection by feeding blood, which are disinfected with 75% ethanol for 3 times with 5 seconds/time, then washed with insect physiological saline for 3 times with 5 seconds/time. The salivary glands are dissected by the tissue technical personnel, and then collected into an EP tube loading with 200 μL of RPMI 1640, the EP tube is kept on ice all the time.

Precautions: 1) If used for direct inoculation, the dissected salivary glands are only needed to be placed into the physiological saline. (2) The salivary glands of mosquitoes are acquired, drawn and beaten for 30-40 times repeatedly with a 18G syringe with a needle to smash the salivary glands and tissues, then centrifuged under 1000 rpm at 0° C. for 5 minutes, the supernatant containing the sporozoites is collected.

Precautions: 1) Intact salivary glands of mosquitoes are acquired as possible when dissected, the collection of tissue fragments is avoided, otherwise the breaking of the salivary glands and the purification of the sporozoites would be affected; 2) it would be best to acquire the sporozoites according to the parasite strains, the acquired sporozoites are labeled to prevent cross-contamination among the parasite strains during the sporozoite acquisition; 3) the process for acquiring the sporozoites should be completed within 1 hour.

### 5. Sporozoite Cryopreservation

(1) A small amount of supernatant containing the sporozoites is taken, and the number of the sporozoites is counted with a cell counting chamber;
(2) the supernatant is centrifuged under 12000 rpm at 0° C. for 10 minutes, the supernatant is discarded, an appropriate amount of CryoStor CS2 or AB type human serum is added to resuspend the precipitate, then the concentration of the sporozoites is adjusted to 2.5*10⁸/mL;
(3) the resuspended sporozoite solution is subpackaged into the frozen vials at 200 μL/tube, labeled, then transferred to liquid nitrogen tank.

### 6. Direct Inoculation of the Sporozoites

(1) 1 ml syringe, 75% alcohol and cotton swabs, etc. are prepared, and volunteers are arranged;
(2) the acquired sporozoite suspension in physiological saline is treated under aseptic conditions, a small amount of which is taken and the number of sporozoites is counted with a cell counting chamber, then the concentration of the sporozoites is adjusted to the number for inoculation;
(3) the volunteers are inoculated via intravenous injection, then isolated in a special ward and observed;
(4) a small amount of sample before inoculation is reserved for sterility test, and records are made;
(5) 3 days post-vaccination, blood smears are made every day for the microscopic examinations of the parasites.
7. Inoculation after the Resuscitation of Sporozoites
(1) 1 ml syringe, 75% alcohol and cotton swabs, etc. are prepared, and volunteers are arranged;
(2) the ultraviolet light is opened to disinfect for 30 minutes or more;
(3) the cryopreserved sporozoites are taken out of the liquid nitrogen tank, then thawed in a water bath kettle at 37° C. for 1-2 minutes;
(4) the thawed sporozoites are centrifuged under 12000 rpm at 0° C. for 10 minutes, the supernatant is discarded, then 10-fold volume of physiological saline is added to resuspend;
(5) the resuspended sporozoites are centrifuged under 12000 rpm at 0° C. for 10 minutes, the supernatant is discarded, then 200 μL of physiological saline is added to resuspend;
(6) the volunteers are vaccinated via intravenous injection, then isolated in a special ward and observed;
(7) a small amount of sample before inoculation is reserved for sterility test, and records are made;
(8) 3 days post-vaccination, blood smears are made every day for the microscopic examinations of the parasites.

The cryopreservng and resuscitating methods of the present invention is beneficial to the infection of the parasites, which can improve the infection efficiency and the effect of the composition.

In a third aspect, the present invention provides a composition as described in the first aspect for the treatment of tumors.

According to the present invention, the composition has therapeutic effects primarily on solid tumors other than melanoma and lymphoma, the tumor includes any solid tumor or a combination of at least two solid tumors of lung carcinoma, gastric carcinoma, colon carcinoma, hepatic carcinoma, or breast carcinoma, etc.

According to the present invention, the treatment of the tumor is that a therapeutic effect can be achieved for the tumor patients successfully infected with the plasmodia, wherein there are many transmission routes for plasmodium infection (malaria), the natural transmission medium is anopheles mosquito, people may be infected after stung by the infectious female anopheles, the plasmodia can be transmitted through blood, malaria can also be transmitted by transfusing blood containing plasmodia or using the syringes comprising the blood containing plasmodia, etc., the specific method of treatment of the present invention comprises the following steps: using the composition as described in the first aspect for a tumor patient by simply transfusing the composition into the body of a tumor patient to successfully infect with plasmodia, which can be performed by those skilled in the art through selecting the method well-known in the art according to the actual needs, preferably through an injection mode.

In the present invention, since it can persist for several years after people were infected with plasmodia, which will develop into chronic plasmodium infection, people also can be repeatedly infected by the same or different species of plasmodium, it can form a long-term repeated plasmodium infection status. Since the composition is transfused to the body of a tumor patient who will suffer from both diseases of plasmodium infection and tumor at the same time, while the plasmodium infection is not lethal, but also can inhibit the growth of tumor cells to extend the life of a tumor patient, which will win for the tumor patient a longer treatment time, and contribute to the treatment and recovery of a patient with tumor.

In the present invention, the plasmodium infection can be used to enhance the immune surveillance mechanism for certain types of solid cancer. During the plasmodium infection, the danger signal molecular pathogens associated pattern recognition molecules (PAMPs) released by the plasmodia can be recognized by the pattern recognition receptors (PRRs) of the host immune cells, including toll-like receptors (TLRs) on the endosome membrane or the cell surface, and RIG-I-like receptors (RLR and NOD-like receptors (NLR)) in the cytoplasm. PAMPs of a plasmodium include the known glycosylphosphatidylinositol anchors (GPI anchors), heme and immunostimulatory nucleic acid motifs and other unknown molecules. PRRs activated by PAMPs of a plasmodium trigger different transcriptional procedures and stimulate multiple downstream signaling pathways to induce systemic immune responses, including release of proinflammatory factors and Thl type cytokines such as TNF-α, IL-1β, IL-2, IL-6, IL-12, type I and type II IFNs, activation of NK cells, NKT cells, γ/δ T cells, macrophages and dendritic cells (DCs), then activation of CD4+ and CD8+T cells, antagonism against tumor immunosuppressive microenvironment containing TGF-β, IL-10, regulatory T cells (Tregs), myeloid-derived suppressor cells (MDSCs) and tumor-associated macrophages (TAMs), such that the immunosuppressive microenvironment will be turned into the immune supportive microenvironment, and ultimately the tumor can be transformed into an effective tumor vaccine. In another aspect, plasmodium infection damage-associated molecular patterns (DAMPs), such as known endogenous uric acid, microbubbles and haem also induce similar immunocompetence. Our previous studies have shown that the blood stage plasmodium infection exhibits antitumor effect by induction of potent antitumor innate immune responses, including secretion of IFN-γ and TNF-α, and activation of NK cells. Plasmodium infection induces adaptive antitumor immunity by increasing tumor-specific T cell proliferation and cytotoxic activity of CD8+T cells (CTL), and increases the infiltration of these cells into tumor tissues to kill the tumor cells.

The inventors also found that the blood stage plasmodium infection can significantly reduce the numbers of Tregs and MDSCs in the tumor tissues of lung carcinoma (LLC)-bearing mice, and reduce the number of TAMs in the tumor tissues of hepatic carcinoma (Hepal-6)-bearing mice, and in addition, the plasmodium infection can significantly inhibit the angiogenesis of tumors in mice.

In the present invention, fever caused by the plasmodium infection may contribute to the death of tumor cells. Parasitemia is necessary for effective inhibition of tumor growth. However, in mice, the plasmodium only causes short-term infection without fever, and it is difficult to observe repeated plasmodium infection in the murine models. Among people lacking effective anti-malarial treatment, plasmodium infection can cause long-term parasitemia accompanied by acute high fever, such syndrome can repeat many times throughout the life period. Therefore, liver stage and blood stage plasmodium infections will be produced by plasmodia naturally acquired by anopheles stinging, which will continuously stimulate the immune system to transform the tumor into an effective tumor vaccine, and act a multi-pathway multi-target therapeutic effect in combination with fever in acute stage and its antiangiogenic effect, etc. In medical literatures, the febrile infection is associated with spontaneous regression of tumors, and malaria is a typical febrile infection.

Compared with the prior art, the present invention has the following beneficial effects:

(1) The composition of the present invention has therapeutic effects on various solid tumors such as colorectal carcinoma, lung carcinoma, breast carcinoma, gastric carcinoma and hepatic carcinoma, etc., and can inhibit the growth of tumor and prolong the life of the patients, whereas has no therapeutic effect on melanoma and lymphoma;
(2) the present invention describes that the long-term plasmodium infection has better therapeutic effect on tumors, and the plasmodium immunotherapy of the present invention does not take the fever time as a course standard when treating tumors, but should be used to extend the duration of plasmodium infection as much as possible until the progression of tumors can be controlled under the premise of protecting the organ functions and life safety of the patients;
(3) The plasmodium immunotherapy (plasmodium infection) of the present invention is relatively economical and safe, while the standard chemotherapy commonly used clinically will often cost more than RMB 20,000-30,000, local radiotherapy for each treatment course will also needs RMB 30,000-50,000, or the targeting drugs (Iressa and Tarceva) will cost about RMB 600 a day, and continuous medication is required which is quite expensive. In contrast, the cost of plasmodium immunotherapy is relatively much lower, and the side effect of which is relatively less, which only needs simple symptomatic treatment, regular monitoring of routine blood and liver and kidney functions, no additional cost will be borne by the patients, moreover the patients can be re-treated or the treatment can be terminated at any time based on the changes in conditions, the treatment course of which can be artificially controlled; for patients who have been suffered from both mental and economic shocks, it is a relatively safe, economical and practical therapeutic option. Therefore, the plasmodium immunotherapy of the present invention is particularly suitable for popularization and application in economically underdeveloped and underserved areas, and the majority of the third world developing countries, which can not only relieve patient's burden, but also combine the advantages of immunotherapy, fever therapy and anti-angiogenic therapy.

### DESCRIPTION OF THE DRAWINGS

FIG. 1 shows the effect of Py17XNL plasmodium (Py) infection on the survival time of tumor-bearing mice, wherein, FIG. 1(A) is the lung carcinoma (LLC) tumor-bearing mice, FIG. 1(B) is the colon carcinoma (C26) tumor-bearing mice, and FIG. 1(C) is the breast carcinoma (EMT6) tumor-bearing mice.

FIG. 2 shows the effect of Py17XNL plasmodium (Py) infection on the survival time of tumor-bearing mice, wherein, FIG. 2(A) is the melanoma (B16) tumor-bearing mice, and FIG. 2(B) is the lymphoma (A20) tumor-bearing mice.

FIG. 3 shows the effect of Py17XNL plasmodium (Py) infection on the survival time of the hepatic carcinoma tumor-bearing mice, wherein, FIG. 3(A) is the Hepal-6 hepatic carcinoma tumor-bearing mice, and FIG. 3(B) is the H22 hepatic carcinoma tumor-bearing mice.

FIG. 4 shows the infection rates of P. chaubaudi (Pc) and Py17XNL Plasmodium yoelii (Py) to tumor-bearing mice.

FIG. 5 shows the growth curves of the lung carcinoma (LLC) tumor-bearing mice infected with P. chaubaudi (Pc) and Py17XNL Plasmodium yoelii (Py).

FIG. 6 shows the changing curves of infection rates of the lung carcinoma (LLC) tumor-bearing mice after the intervention of chloroquine phosphate (CQ) in the plasmodium infection.

FIG. 7 shows the effect of the intervention of chloroquine phosphate (CQ) in the time course of plasmodium infection on the survival time of lung carcinoma (LLC) tumor-bearing mice.

FIG. 8 shows the prolonged survival time of mice with surgical removal of the tumor by plasmodium infection.

FIG. 9 shows the reduction of the number of Tregs cells in tumor tissues by plasmodium infection.

FIG. 10 shows the reduction of the number of MDSCs cells in tumor tissues by plasmodium infection.

FIG. 11 shows the effect of plasmodium infection on infiltration of TAMs in tumor-bearing mice.

### SPECIFIC EMBODIMENTS

In order to further set forth the technical means adopted by the present invention and the technical effects thereof, the technical solutions of the present invention will be further illustrated by the specific embodiments with reference to the accompanying drawings, but the present invention is not limited to the scope of the embodiments.

### Experimental Materials

(1) 6-8-week-old of female C57BL/6 mice and Balb/c mice were purchased from Shanghai Slac Laboratory Animal Co., Ltd. or Beijing Vital River Laboratory Animal Technology Co., Ltd.
(2) Mouse P. chabaudi ASS (MRA-429, Pc) and mouse P. yoelii 17XNL (MRA-593, Py) were both from Malaria Research and Reference Reagent Resource Center (MR4) as complimentary gifts.
(3) Mouse colon carcinoma cell line C26, breast carcinoma cell line EMT6, mouse Lewis lung carcinoma cell line LLC, hepatic carcinoma cell line Hepal-6 and H22, and melanoma cell line B16 were purchased from cell bank of Chinese Academy of Sciences (Shanghai).
(4) Mouse lymphoma cell line A20 was purchased from American Type Culture Collection (ATCC).
(5) Chloroquine phosphate (chloroquine, CQ) was a product manufactured by Sigma-Aldrich Corporation.
(6) Glycerin (C3H803) was from Zhejiang Suichang Huikang Pharmaceutical Co. Ltd.
(7) Sorbitol (C6H1406) was from Shijiazhuang Ruixue Pharmaceutical Co. Ltd.
(8) Sodium chloride injection was from Chenxin Pharmaceutical Co., Ltd.
(9) Giemsa stain powder was from Sigma-Aldrich Corporation.

### EXAMPLE 1 SIGNIFICANTLY NEGATIVE CORRELATIONS BETWEEN INCIDENCE OF MALARIA WITH TOTAL MORTALITY OF TUMOR AND MORTALITIES OF COLORECTAL CARCINOMA, LUNG CARCINOMA, BREAST CARCINOMA AND GASTRIC CARCINOMA

Malaria cases reported by the WHO were used: data sources of the reported malaria cases: 1955-1964, WHO Epidemiological and Vital Statistics Report (1966); 1962-1981, WHO World Health Statistics Annual (1983); 1982-1997, WHO Weekly Epidemiological Record (1999); and 1990-2008, WHO 2009 annual report. Reported malaria cases of 218 countries in 1955-2008 were obtained based on the above reports. Population data of 228 countries during the period of 1955-2008 were obtained from the U.S. Census Bureau International Database (http://www.census.gov). Calculation of the incidence of malaria: the incidence data of malaria of 170 countries were obtained from the reported number of malaria cases divided by the total population of the country.

Mortality data of tumor: Age standardized mortality data, mortality data of total cancers and 29 cancers (1955-2008) were obtained from the cancer mortality database of WHO (http://www-dep.iarc.fr/WHOdb/WHOdb.htm), and classified according to gender. Incidence data of malaria and mortality data of cancer of 56 countries in 1955-2008 were obtained when combined with the incidence data of malaria. Income information of these 56 countries was obtained from the World Bank database (http://data.worldbank.org/country). Population life expectancy data for these 56 countries (both male and female) and general-geographic data were from the information published by Population Division of Department of Economic and Social Affairs of the United Nations (World Population Prospects: The 2012 Revision; http://esa.un.org/wpp/).

Epidemiological data analysis was carried out according to the above malaria cases and mortality data of tumor reported by WHO: the trends of incidence of malaria and mortality of cancer were analyzed by linear regression, then the correlation between the incidence of malaria and mortality of cancer was detected using the generalized additive mixed model (GAMM), the adjusted confounding factors include the income level, life expectancy and geographical location of the state. All analyses were carried out using the authorised software (www.empowerstats.com, X&Y solutions, Inc., Boston, Mass.) and R software (http://www.R-project.org), the correlation between the mortality of 30 tumors and the incidence of malaria in 56 countries from 1955 to 2008 was analyzed after correction of regional distribution, income level, life expectancy, time, and trend of incidence of malaria, the results were shown in Table 1 and Table 2.

TABLE 1
Regression analysis between tumor mortality resulted
from all causes and malaria incidence in 1955-2008

| | Male | Female |
|---|---|---|
| Basic model | −0.031 (−0.037, | −0.032 (−0.037, |
| | −0.025) *** | −0.026) *** |
| Correction model | −0.020 (−0.027, | −0.020 (−0.025, |
| | −0.014) *** | −0.014) *** |
| Time period | | |
| 1955-1981 | −0.011 (−0.016, | −0.005 (−0.010, |
| | −0.005) *** | 0.000) ** |
| 1982-2008 | −0.025 (−0.034, | −0.011 (−0.019, |
| | −0.015) *** | −0.003) *** |
| Income | | |
| Low | −0.028 (−0.037, | −0.025 (−0.034, |
| | −0.018) *** | −0.016) *** |
| High | −0.011 (−0.015, | −0.007 (−0.010, |
| | −0.006) *** | −0.003) *** |
| Trends of Incidence | | |
| of Malaria | | |
| decreasing | −0.020 (−0.031, | −0.025 (−0.036, |
| | −0.008) *** | −0.013) *** |
| No obvious change | −0.013 (−0.026, | −0.006 (−0.018, |
| | 0.000) * | 0.006) |
| increasing | −0.021 (−0.032, | −0.003 (−0.012, |
| | −0.010) *** | 0.007) |

Note:
The numerical values in the table were regression coefficients; both malaria incidence and tumor mortality had been through log conversions.
*** p < 0.001;
** p < 0.01;
* p < 0.05

TABLE 2
Regression Analysis of Single Tumor Mortality
and Malaria Incidence in 1955-2008

| Gender | Basic model | Correction model |
|---|---|---|
| Colon Carcinoma | | |
| Male | −0.072 (−0.087, | −0.052 (−0.068, |
| | −0.056) *** | −0.036) *** |
| Female | −0.087 (−0.102, | −0.063 (−0.078, |
| | −0.072) *** | −0.048) *** |
| Colorectal carcinoma | | |
| and Anal carcinoma | | |
| Male | −0.051 (−0.062, | −0.037 (−0.047, |
| | −0.041) *** | −0.026) *** |
| Female | −0.058 (−0.068, | −0.042 (−0.052, |
| | −0.048) *** | −0.032) *** |
| Lung Carcinoma | | |
| Male | −0.036 (−0.046, | −0.020 (−0.032, |
| | −0.025) *** | −0.009) *** |
| Breast Carcinoma | | |
| Female | −0.046 (−0.056, | −0.030 (−0.040, |
| | −0.037) *** | −0.020) *** |
| Gastric Carcinoma | | |
| Male | −0.066 (−0.076, | −0.039 (−0.050, |
| | −0.055) *** | −0.028) *** |

Note:
The numerical values in the table were regression coefficients; both malaria incidence and tumor mortality had been through log conversions.
*** p < 0.001;
**p < 0.01;
*p < 0.05

As can be seen from the results in Table 1 and Table 2, total tumor mortalities were significantly negatively correlated with malaria incidences, such negative correlations were present in both male and female, and high-income and low-income countries, and the significant negative correlations were present in countries with different trends of malaria incidences (increasing, decreasing and no obvious change). Further analysis found that there were significant negative correlations between the mortalities of 5 main tumors such as colorectal carcinoma (male and female), colon carcinoma (male and female), lung carcinoma (male), breast carcinoma (female) and gastric carcinoma (male) and the malaria incidence. These results suggest that plasmodium infection, i.e. malaria, may have the potential to treat solid tumors.

### EXAMPLE 2 CRYOPRESERVATION AND RESUSCITATION OF PLASMODIUM BLOOD

After the plasmodium blood was put in storage, it should be subpackaged as soon as possible according to the requirements, the subpackaged plasmodium blood should be labeled as required, and preserved in liquid nitrogen.

Preparation of the experimental materials: peripheral blood of malaria patients without other statutory blood-borne infectious disease, or plasmodium blood cultured in laboratory. Sodium chloride injection, 10% NaCl solution, 3.5% NaCl solution, RPMI 1640 culture medium, 28% glycerin cryopreservation solution, cryopreservation tube, 50 mL centrifuge tube and Pasteur tube, etc.

28% glycerin cryopreservation solution, the components of which are:

28% glycerin cryopreservation solution,
each 100 mL of which contains:

| | |
|---|---|
| Glycerin (C₃H₈O₃) | 28 g |
| Sorbitol (C₆H₁₄O₆) | 3 g |
| Sodium chloride injection | Which is diluted to 100 mL |

### 1. Cryopreserving the Plasmodia:

(1) Previous preparations: sterilized scissors, 500 cryopreservation tubes, 50 mL centrifuge tubes, 15 mL centrifuge tubes, 250 mL culture medium bottles, sodium chloride injection, 1640 culture medium, and 28% glycerin cryopreservation solution were prepared, the labels, SOP and lists were printed, and the liquid nitrogen tank was checked, etc.;
(2) an appropriate amount of samples of A, B or C plasmodium blood were taken, the blood smears of which were made for microscopic examination, the cryopreserved plasmodia were the ones of which 30% or more were plasmodia in the period of early trophozoites, and the microscopic examination reports were filled in;
Notes: 200 mL plasmodium blood can be treated in batches to reduce the number of samples to be treated at the same time; 50 mL centrifuge tubes: 10 mL/tube*20 tubes; 17 tubes were placed at 4° C. to temporarily store; 10 mL*3 tubes, centrifuged to remove the white blood cells, washed with 1640 culture medium once, cryopreservation solution was added, and subpackaged at 1 mL/tube, stood still at room temperature for 5 minutes, then placed into the liquid nitrogen; likewise, 17 tubes*15 mL were treated again.
(3) the obtained in step (2) was centrifuged at 300 g (1200 rpm) for 10 min (with speed up and down of 1), then the plasma was transferred to another 50 mL centrifuge tube, followed by subpackaging at 1 mL/tube and cryopreserving;
(4) the layer of white blood cell was drawn out, 2-fold volume of 1640 culture medium was added, then mixed well, the mixture was centrifuged at 300 g for 5 minutes (with speed up and down of 1, 1200 rpm);
(5) the 50 mL centrifuge tube was placed on a vortex shaker, and an equal volume of 28% glycerin cryopreservation solution (7-8 mL) was added dropwise to a 50 mL centrifuge tube while shaken to mix well, then incubated for 5 minutes at room temperature;
(6) the RBC-28% glycerin cryopreservation solution mixture was subpackaged into cryopreservation tubes with 1.0 mL per tube;
(7) the cryopreservation tubes were put into a cryopreservation box, which was preserved in liquid nitrogen directly.

Quality control of cryopreservation: an appropriate amount of RBC-28% glycerin cryopreservation solution mixture before cryopreservation was taken and an appropriate amount of RPMI 1640 culture medium was added thereto, the mixture was cultured at 37° C. and incubated in a carbon dioxide incubator for 72h, then the color of the culture medium was observed to confirm no turbidity.

### 2. Resuscitating the Plasmodia:

(1) advance preparations: a water bath kettle (37° C.), a 50 mL centrifuge tube, 3.5% NaCl, sodium chloride injection, a centrifuge were prepared, and labels and lists, etc., were printed; (2) the labels of the cryopreservation tubes, etc. were checked;
(3) the cryopreserved blood of the parasite was taken (1.0 mL/tube), and placed into a water bath at 37° C. for 1-3 minutes to be thawed;
(4) the thawed blood of the parasite was transferred to a 15 mL centrifuge tube and an equal volume of 3.5% NaCl was slowly added along the wall of the tube, which was gently blown and beaten to mix well, then stood still at room temperature for 5 minutes, centrifuged at 300 g for 5 minutes, and the supernatant was discarded;
(5) 5-fold volume of 0.9% NaCl was slowly added along the wall of the tube, which was gently blown and beaten to mix well, centrifuged at 300 g for 5 minutes, and the supernatant was discarded;
(6) physiological saline was added until the hematocrit (HCT) reached 50%, mixed well, the density of the red blood cell was counted and recorded, temporarily stored at 4° C. to be used for clinical vaccination.

Quality control of resuscitation: after the vaccination, a blood smear of the remaining blood sample was prepared, and then observed by microscopy for the infection rate and the status of the parasite before the injection, and records were made.

The temperature of refrigerator for preserving the plasmodium blood should be monitored regularly, and records were made.

The level of liquid nitrogen in the liquid nitrogen tank should be checked regularly, the liquid nitrogen should be added timely, and records were made. Ex-warehouse and in-warehouse of plasmodium blood should be ensured to be recorded on the required forms and the quantities of which must be accurate.

### EXAMPLE 3 CRYOPRESERVATING, RESUSCITATING AND VACCINATION PROCEDURES OF PLASMODIUM SPOROZOITES 1. BLOOD-FEEDING OF THE ANOPHELES

(1) Previous preparations: the density of the protozoa and the gametophyte condition of the volunteers with malaria were monitored, 2 ml of peripheral blood was collected via vein, put into a thermos flask immediately, transported to the anopheles' house as soon as possible (within 2 hours), and the anopheles were starved 24 hours in advance (300 or more of anopheles);
(2) before feeding blood, the room temperature of the anopheles' house was adjusted to 26° C., the membrane blood-feeding system was made ready, and the collected peripheral blood was added to the membrane blood-feeding system, and feeding blood for 30 minutes;
(3) the anopheles not satiate were drawn out, labeled, and placed into an incubator at 26° C. to feed, then a sugar water cotton containing 10% glucose+0.05% PABA was added;
(4) the temperatures before and after transportation of the thermos flask, the time of blood-feeding, the number of anopheles and the blood-feeding situation of anopheles were recorded.

### 2. Oocyst Examination

(1) The oocysts were examined at day 7-10 after the infection by feeding blood, and 10 oocysts were dissected to calculate the proportion of positive anopheles;
precautions: 1) infectiosity standard of oocyst, +: 1-10, ++: 11-100, and +++: 101 or more;
(2) if the ratio of “++” or more of the infectiosity of oocyst was <50%, 20 anopheles were reexamined.

### 3. Sporozoite Examination

(1) The sporozoites were examined at day 14-16 after the infection by feeding blood, 20 anopheles were dissected, and the total number of sporozoites and the average number of sporozoites per anophele were calculated;
Precautions: 1) A cell counting chamber was used for counting, the number of all sporozoites in the big grid was counted, and followed the principle of “counting the upper rather than the lower, and counting the left rather than the right”.
(2) an average value of three times of counting were taken to minimize the errors when counting.

### 4. Sporozoite Acquisition

(1) The anopheles were taken out at day 14-20 after the infection by feeding blood, which were disinfected with 75% ethanol for 3 times with 5 seconds/time, then washed with insect physiological saline for 3 times with 5 seconds/time. Salivary glands were dissected by the tissue technical personnels, and then collected into an EP tube loading with 200 mL of AB+human serum (Sigma), the EP tube was kept on ice all the time.
Precautions: (1) If used for direct vaccination, the dissected salivary glands were only needed to be placed into the physiological saline; (2) the salivary glands of anopheles were acquired, drawn and beaten for 30-40 times repeatedly with a 18G syringe with a needle to smash the salivary glands and tissues, then centrifuged under 1000 rpm at 0° C. for 5 minutes, the supernatant containing the sporozoites was collected.
Precautions: 1) The intact salivary glands of anopheles were acquired as possible when dissected, the collection of tissue fragments was avoided, otherwise the breaking of the salivary glands and the purification of the sporozoites would be affected; 2) it would be best to acquire the sporozoites according to the parasite strains, the acquired sporozoites were labeled to prevent cross-contamination among the parasite strains during the sporozoite acquisition; 3) the process for acquiring the sporozoites should be controlled within 1 hour.

### 5. Sporozoite Cryopreservation

(1) A small amount of above supernatant containing the sporozoites was taken, and the number of the sporozoites was counted with a cell counting chamber;
(2) the supernatant was centrifuged under 12000 rpm at 0° C. for 10 minutes, the supernatant was discarded, an appropriate amount of AB type human serum containing 1% tri-antibody was added to resuspend the precipitate, then the concentration of the sporozoites was adjusted to 2.5*10⁸/mL;
(3) the resuspended sporozoite solution was subpackaged into the cryopreservation tubes at 200 μL/tube, labeled, then placed into alcohol at −80° C. to quick-freeze, transferred to liquid nitrogen tank 3 hours later.

### 6. Direct Vaccination of the Sporozoites

(1) 1 ml syringe, 75% alcohol and cotton swabs, etc. were prepared, and volunteers were arranged;
(2) the acquired sporozoite suspension in physiological saline was treated under aseptic conditions, a small amount of which was taken and the number of sporozoites was counted with a cell counting chamber, then the concentration of the sporozoites was adjusted to the number for vaccination;
(3) the volunteers were vaccinated via intravenous injection, then isolated in a special ward and observed;
(4) a small amount of sample before vaccination was reserved for sterility test, and records were made;
(5) 3 days post-vaccination, blood smears were made every day for the microscopic examinations of plasmodia.
7. Vaccination after the Resuscitation of Sporozoites
(1) 1 ml syringe, 75% alcohol and cotton swabs, etc. were prepared, and volunteers were arranged;
(2) the ultraviolet light was opened to disinfect for 30 minutes or more;
(3) the cryopreserved sporozoites were taken out of the liquid nitrogen tank, then thawed in a water bath kettle at 37° C. for 1-2 minutes;
(4) the thawed sporozoites were centrifuged under 12000 rpm at 0° C. for 10 minutes, the supernatant was discarded, then 10-fold volume of physiological saline was added to resuspend;
(5) the resuspended sporozoites were centrifuged under 12000 rpm at 0° C. for 10 minutes, the supernatant was discarded, then 200 μl of physiological saline was added to resuspend;
(6) the volunteers were vaccinated via intravenous injection, then isolated in a special ward and observed;
(7) a small amount of sample before vaccination was reserved for sterility test, and records were made;
(8) 3 days post-vaccination, blood smears were made every day for the microscopic examinations of plasmodia.

### EXAMPLE 4 OBSERVATION OF THERAPEUTIC EFFECTS OF PLASMODIUM INFECTION ON LUNG CARCINOMA, COLON CARCINOMA, BREAST CARCINOMA, MELANOMA AND LYMPHOMA TUMOR-BEARING MICE

According to the results of the epidemiological analysis, animal experiments on 5 tumor models such as lung carcinoma, colon carcinoma, breast carcinoma, melanoma and lymphoma (due to lack of appropriate animal model, gastric carcinoma cannot be tested) and benign plasmodium (Py17XNL, Plasmodium yoelii non-lethal strain) infection in mice were carried out to observe whether plasmodium infection can improve the survival time and survival rate of tumor-bearing mice.

The specific experimental steps were as follows:

(1) Preparation of mouse model: 6-8-week-old of Balb/c mice were subcutaneously injected with 2×10⁵ of C26 cells (colon carcinoma cell line) and 1×10⁶ of A20 cells (lymphoma cell line), and breast pad injected with 5×10⁵ of EMT6 cells (breast carcinoma cell line). 8-10-week-old of C57BL/6 mice were subcutaneously injected with 5×10⁵ of B16 cells (melanoma cell line), and 5×10⁵ of LLC cells (Lung carcinoma cell line).

The mice were intraperitoneally injected with 5×10⁵ of Py17XNL infected red blood cells: Py+C26, Py+A20, Py+EMT6, Py+B16, and Py+LLC as the treatment group at the same time with the inoculation of tumor cells, the mice were given with simple tumor cells, meanwhile intraperitoneally injected with the same number of normal red blood cells of mice as the corresponding control group: C26, A20, EMT6, B16, and LLC.

(2) Treatment: Spontaneous death of mice or euthanasia of the mice with dying signs were used as the end point of survival, there was no accidental death during the experiment, the animals were randomly divided into cages to observe whether plasmodium infection can improve the survival time and survival rate of tumor-bearing mice, and the survival situations of tumor-bearing mice were recorded.

(3) Experimental results

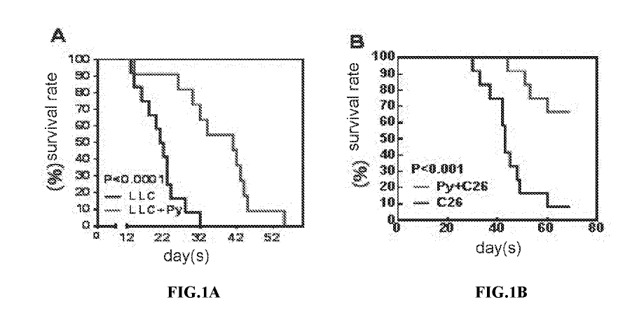

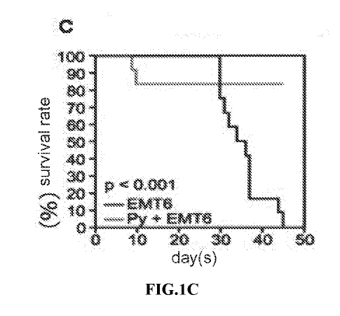

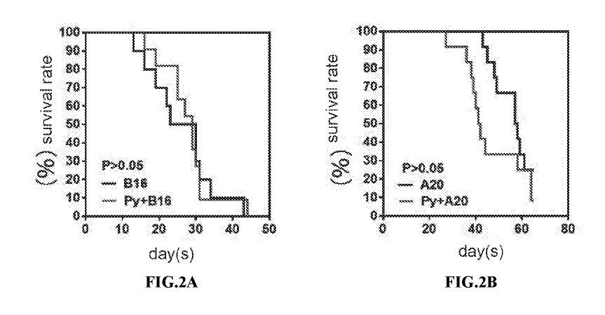

The experimental results of lung carcinoma, colon carcinoma, and breast carcinoma in mice were as shown FIG. 1(A), FIG. 1(B) and FIG. 1(C), and the experimental results of melanoma and lymphoma in mice were as shown in FIG. 2(A) and FIG. 2(B). The results showed that the benign plasmodium infection had significantly prolonged the median survival time of mice bearing tumor selected from one or more of lung carcinoma, colon carcinoma, and breast carcinoma, which was consistent with the result of epidemiological analysis in Example 1; however, plasmodium infection had no significant influence on the survival time of melanoma (B16) and lymphoma (A20) tumor-bearing mice, which was also consistent with the analysis result of little correlation between the mortalities of melanoma and lymphoma and the incidence of malaria in the epidemiological analysis in Example 1.

### EXAMPLE 5 EFFECT OF PLASMODIUM INFECTION ON SURVIVAL TIME OF HEPATIC CARCINOMA TUMOR-BEARING MICE

We had imitated the method in Example 4, the mice were subcutaneously inoculated with 1×10⁶ of H22 cells (hepatic carcinoma cell line), and 1×10⁶ of Hepal-6 cells (hepatic carcinoma cell line), respectively, the mice were intraperitoneally inoculated with 5×10⁵ of plasmodium infected red blood cells/mouse as the treatment group Py+H22, and Py+Hepal-6; or intraperitoneally injected with 5×10⁵ of uninfected red blood cells in mice/mouse (with inoculation volume of 0.2 mL) as the corresponding control group H22, Hepal-6 (simple tumor group).

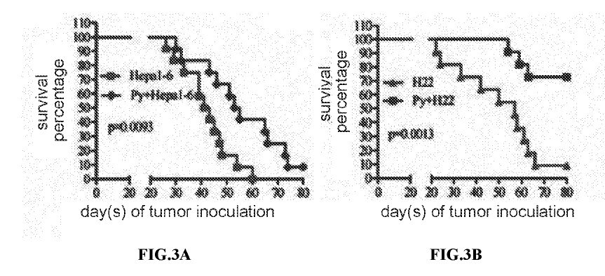

The results were as shown in FIGS. 3(A) and (B), it can be seen that the plasmodium infection had significantly prolonged the survival time of the hepatic carcinoma tumor-bearing mice.

### EXAMPLE 6 EFFECTS OF LONG-TERM PLASMODIUM INFECTION AND SHORT-TERM PLASMODIUM INFECTION ON TUMORS

Subcutaneous transplantation model of mouse Lewis lung carcinoma was used: in order to establish the subcutaneous model of Lewis lung carcinoma, 5×10⁵ of LLC cells/mouse was subcutaneously inoculated in the lateral right shoulder of mice, meanwhile the mice were intraperitoneally injected with 5×10⁵ of P. chaubaudi (Pc) infected red blood cells/mouse (LLC+Pc group), or meanwhile the mice were intraperitoneally injected with 5×10⁵ of P. yoelii (Py) infected red blood cells/mouse (LLC+Py group), and the mice without inoculation of tumor cells were intraperitoneally injected with 5×10⁵ of Pc or Py infected red blood cells/mouse as the control groups (referred to as Pc group and P group, respectively).

Staining of blood film: The thin film was stained after fixed with methanol, while the thick film was stained directly, the 10×Giemsa staining solution (5 g/L Giemsa stain powder, 25% Glycerin, and 25% Methanol) was diluted with PBS to a 1×Giemsa staining solution to stain for 30-60 minutes, the staining solution was washed off with tap water, blow-dried, then the infection rate was observed with oil microscopy (100×).

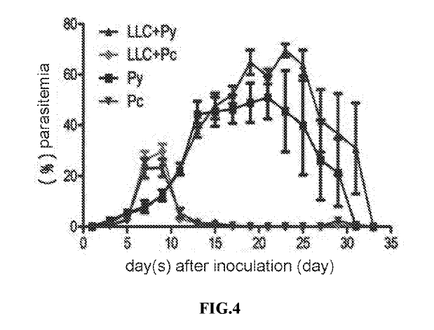

The results were as shown in FIG. 4, it can be seen that there were differences in infection time course and pathogenicity among plasmodium strains in mice, the natural cycle (time course) of short-term plasmodium infection (Pc) was 2 weeks, the infection rate reached its peak of about 30% on day 7 after infection, followed by declining rapidly. The natural cycle of long-term plasmodium infection (Py) was about 1 month, the infection rate reached its peak at about week 2, followed by maintaining at high infection rate for a period of time, then beginning to decline on about day 20, and the protozoa disappeared on about day 30. Both of the above plasmodia were benign plasmodia, with which the infected C57BL/6 mice would not die. When compared the LLC+Pc group with the Pc group, the infection rates of both were similar; however, the protozoa infection rate (peak) of the LLC+Py group was slightly higher than that of the Py group, and the cycle was also slightly longer. In general, however, the inoculation of tumor had no significant influence on the infection time course of the plasmodium.

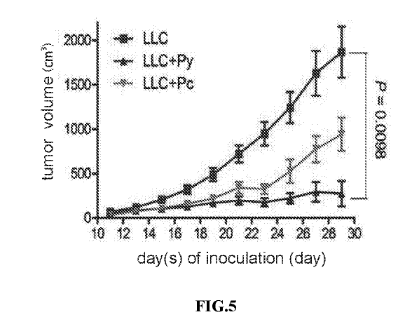

The antitumor effects of both plasmodia were as shown in FIG. 5, the results showed that the malignancy of LLC lung carcinoma was higher and the growth rate of which was faster. However, both short-term P. chaubaudi (Pc) infection and the relatively long-term P. yoelii (Py) infection could significantly inhibit the growth of tumor. It was found that the effect of Py infection on tumor growth was significantly better than that of Pc infection, thus it can be seen that the infection time course of Plasmodium may be one of the parameters that affect the tumor growth: the longer the infection time course, the more obvious inhibition of the tumor growth.

### EXAMPLE 7 EFFECT OF PLASMODIUM INFECTION TIME COURSE ON SURVIVAL TIME OF TUMOR-BEARING MICE

The mice were injected with 5×10⁵ of LLC cells/mouse via caudal vein, and intraperitoneally inoculated with 5×10⁵ of P. yoelii (Py) infected red blood cells/mouse as the treatment group, or the LLC inoculated mice were intraperitoneally injected with 5×10⁵ of uninfected red blood cells/mouse as the non-treatment control group, all the inoculation volumes were 0.2 mL. The treatment group was further divided into four groups after inoculation of tumor and plasmodium. The plasmodium infections of the first three groups were terminated with chloroquine (CQ) phosphate on day 7, 14 and 21, and the fourth group did not use chloroquine and let the plasmodium infection to naturally terminate and acted as a whole-course plasmodium infection group, the above four groups were labeled as LLC+Py+d7 CQ group, LLC+Py+d14 CQ group, LLC+Py+d21 CQ group and LLC+Py group, respectively, the control group with simple tumor without infection with plasmodium but treated with CQ was labeled as LLC+CQ group, the control group with simple tumor without infection with plasmodium and not treated with CQ was labeled as LLC group, the control group with simple inoculation of plasmodium but without inoculation of LLC and not treated with CQ was labeled as Py group. The survival times of mice in different groups were compared. The mice were observed on a daily basis, meanwhile blood was taken via caudal vein every two days after inoculation with plasmodium, and made into blood smear, then the infection rate of the plasmodium inoculation group was observed under an oil microscopy after Giemsa staining.

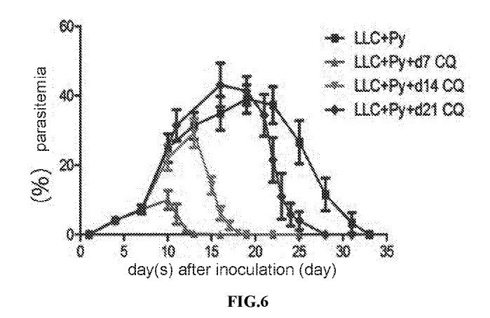

The results were as shown in FIG. 6, chloroquine (CQ) is a specific drug for the treatment of plasmodium infection, the parasitemia would decrease rapidly after administrated with chloroquine on day 7, day 14 and day 21, and the plasmodium were not detected in peripheral blood smears on day 13, day 19 and day 28, indicating that CQ could effectively terminate the plasmodium infection.

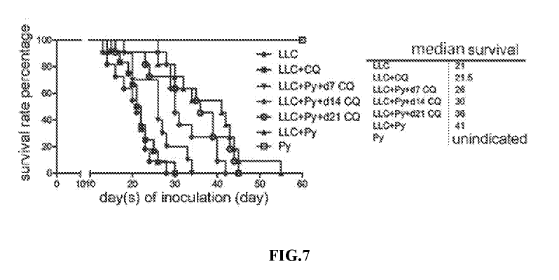

The results were as shown in FIG. 7, CQ treatment did not affect the survival of tumor-bearing mice, and there was no difference in survival time between LLC group and LLC+CQ group, termination of plasmodium infection by CQ would shorten the survival time of tumor-bearing mice, the later the termination of the plasmodium infection was, the longer the time course of the plasmodium infection was, which could prolong the survival time of tumor-bearing mice more significantly. The median survival of tumor-bearing mice also showed that the median survival of mice in the LLC+Py group whose infection was not terminated with CQ was the longest, which was about 2 times longer than that of the simple tumor group (LLC group), the median survival of the mice whose infection was terminated on day 14 was longer than that terminated on day 7, likewise, the median survival of the mice whose infection was terminated on day 21 was longer than that terminated on day 14. In conclusion, the longer the time course of the plasmodium infection is, the more obvious the inhibition of the tumor is, the tumor-bearing mice with longer infection time course had longer survival time.

### EXAMPLE 8 THE PROLONGED SURVIVAL TIME OF MICE WITH SURGICAL REMOVAL OF THE TUMOR BY PLASMODIUM INFECTION

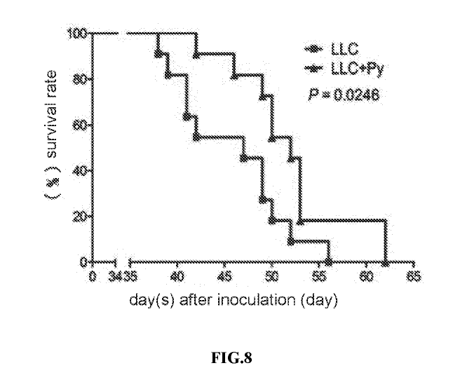

The C57BL/6 mice were subcutaneously inoculated with 5×10⁵ of tumor cells and let the tumor grew for 12 days. The mice was anesthetized, the tumor was surgically removed, and sutured. After another 5 days (let the mice recover), the mice were divided into two groups on day 17, 11 mice for each group. The plasmodium infection group was intraperitoneally inoculated with 5×10⁵ of plasmodium infected red blood cells/0.2 mL, and the control was intraperitoneally inoculated with 5×10⁵ of uninfected red blood cells/0.2 mL, then the survival time of mice was observed. After the subcutaneous tumor was removed, the mice in the LLC group died one after another after 38 days, the results were as shown in FIG. 8. In order to observe the cause of death in mice, the mice were dissected after death, it was found that a large number of tumor nodules were contained in the liver and lung tissues of mice in the LLC group, and these tumor nodules were large in volume. However, the above phenomenon was rare in the LLC+Py group, thus it was thought that the mice in the LLC group were died of the mass growth of metastatic tumor; while the survival cycle of mice in the plasmodium infection group (LLC+Py group) was significantly prolonged, indicating that the plasmodium infection could inhibit the metastasis of cancer cells or kill the metastatic cancer cells or inhibit the growth of the metastatic tumor.

### EXAMPLE 9 SIGNIFICANT DOWN-REGULATION OF THE NUMBER OF MDSCS AND TREGS IN TUMOR TISSUES BY PLASMODIUM INFECTION

At present, a large number of studies have found that a group of myeloid-derived suppressor cells (MDSCs) extensively are present in the spleen, blood and tumor tissue of the tumor-bearing mice or the peripheral blood and tumor tissue of the tumor patients, which can be recruited to the periphery from the bone marrow by tumor-derived factor and further induced activation, promotion of tumor growth can be achieved by inhibiting the proliferation and activation of natural immune macrophages, NK cells and specific immune T cells, which is conducive to tumor metastasis, even the neovascularization of tumor can be promoted by direct insertion of endothelium into the tumor. In addition, MDSCs can promote the formation of regulatory T cells (Tregs) in vivo and in vitro and recruit Tregs into tumor tissues; close contact between Tregs and effector T lymphocytes inhibits the function of effector cells, plays its immunosuppressive effect, which makes the tumor escape the immune surveillance and attack from the body, and promotes the development of tumor.

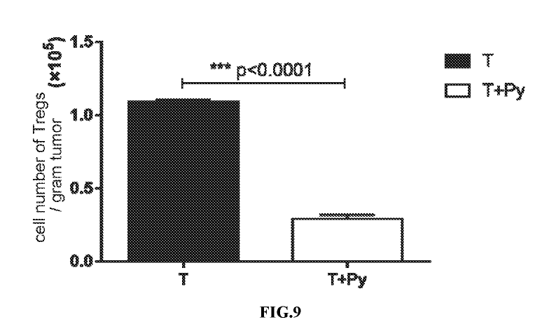

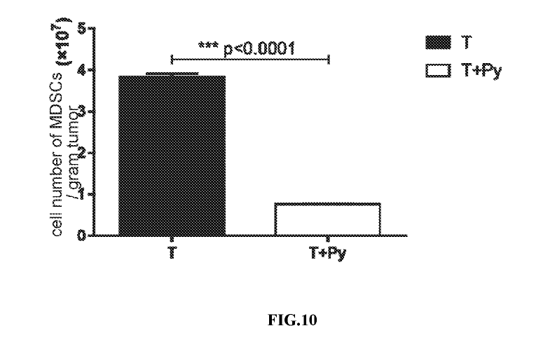

The C57BL/6 mice were subcutaneously inoculated with 5×10⁵ of LLC cells on day 0, and divided into 2 groups: simple tumor control group (T) was intraperitoneally injected 5×10⁵ of uninfected red blood cells on day 7, and the tumor plus the plasmodium infection group (T+Py) was intraperitoneally inoculated with 5×10⁵ of Py infected red blood cells on day 7. Fifteen days after the tumor inoculation, the tumor infiltrating cells were isolated, and staining with fluorescent antibody was performed on MDSCs (Anti-mouse CD8, Anti-Mouse CD11b, and Anti-Mouse Ly-6G (Gr-1)) and Tregs (CD8, CD4, and CD25 antibodies surface staining, and FoxP3 intracellular staining), then analyzed on a flow cytometer, the results were as shown in FIG. 9 and FIG. 10.

As can be seen from FIG. 9 and FIG. 10, plasmodium infection significantly reduced the number of Tregs cells and MDSCs cells in the tumor tissues and improved the immunosuppressive microenvironment of the tumor.

### EXAMPLE 10 SIGNIFICANT DOWN-REGULATION OF TUMOR-ASSOCIATED MACROPHAGES (TAMS) IN THE TUMOR TISSUES BY PLASMODIUM INFECTION

Macrophage is one of the major inflammatory cells infiltrated in tumor body of most solid tumors. Studies have shown that tumor-associated macrophages (TAMs) are one of the key regulatory factors for angiogenesis of solid tumors. Under the action of tumor microenvironment, TAMs were polarized to replace the activated phenotypic feature (M2) and promote the tumor angiogenesis by secreting immunosuppressive cytokinesis, chemokines, and angiogenic factors, etc. to nourish tumor growth. The infiltration level of TAMs was positively correlated with tumor progression. If the level of TAMs infiltration was reduced, the tumor progression can be delayed.

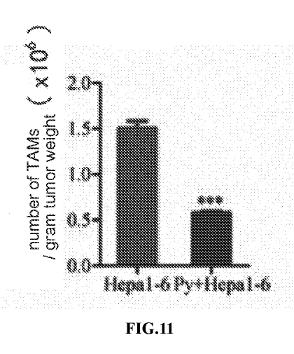

Mice were inoculated with tumor cells and plasmodia in the same way as that in Example 5, Hepal-6 hepatic carcinoma tumor-bearing mice were sacrificed by cervical vertebra dislocation after 17 days of tumor inoculation. The tumor tissues were peeled off and the TAMs were isolated and prepared by the method described by Kusmartsev, et al. The proportion of each subset of cells was labeled by anti-F4/80 antibody and analyzed by FACS Aria flow cytometer. Plasmodium infected and uninfected Hepal-6 hepatic carcinoma tumor-bearing mice and the proportion and relative number of TAMs infiltration in the tumor tissues after 17 days of tumor inoculation were examined with flow cytometry, the results were as shown in FIG. 11: compared with the simple tumor group, the plasmodium infection significantly reduced the number of TAMs in the tumor tissues of Hepal-6 hepatic carcinoma tumor-bearing mice (***P<0.001).

In conclusion, for the model mice, Py with the longest infection period can only last for about one month, subsequently the body will completely remove the plasmodia. At the same time, the mice cannot repeatedly infected with a plasmodium, that is, the mice infected with a plasmodium cannot be infected with homologous and heterologous plasmodia again. However, in humans, the plasmodia can survive for several years after infection, which will develop a chronic plasmodium infection. While our experiment found that the longer the infection period of plasmodium is, the more obvious the inhibition effect on tumor is. In addition, the study also found that plasmodium infection in mice did not cause fever in mice, thus the anti-tumor effect caused by plasmodium infection in mice was not due to the thermotherapy effect caused by fever. While the plasmodium infection in human can cause periodic high fever in acute stage, which meanwhile may also play a thermotherapy effect on the tumor. Therefore, it can be predicted that the effect of plasmodium infection on inhibition of human tumors may be more significant.

Applicant has stated that although the detailed methods of the present invention have been described by the above examples in the present invention, the present invention is not limited to the detailed methods described above, that is to say, it is not meant that the present invention has to be implemented depending on the above detailed methods. It will be apparent to those skilled in the art that any improvements made to the present invention, equivalent replacements to the raw materials and addition of adjuvant ingredients of the products of the present invention, and choices of the specific implementations, etc., all fall within the protection scope and the disclosure scope of the present invention.

1. A composition comprising a plasmodium.

2. The composition according to claim 1, wherein the plasmodium is any one or a mixture of at least two of P. falciparum, P. vivax, P. malariae, P. ovale or P. knowlesi.

3. The composition according to claim 1, wherein the plasmodium causes a long-term plasmodium infection.

4. The composition according to claim 1, wherein the long-term plasmodium infection is a plasmodium infection lasting to a chronic phase of malaria, with an infection time course of more than 6-8 weeks.

5. The composition according to claim 1, wherein the inoculation amount of the plasmodium is not less than 100 plasmodium infected red blood cells or not less than 5 plasmodium sporozoites.

6. The composition according to claim 5, wherein the composition further comprises a pharmaceutically acceptable adjuvant.

7. A method for preparing the composition according to claim 1, wherein the method comprises the following steps:
cryopreserving and resuscitating the plasmodia or the plasmodium sporozoites, and preparing the plasmodia or the plasmodium sporozoites into the composition.

8. A method of treating a tumor, comprising administering to a patient in need thereof a therapeutically effective amount of the composition according to claim 1.

9. The method according to claim 8, wherein the tumor is any solid tumor or a combination of at least two solid tumors selected from the group consisting of lung carcinoma, hepatic carcinoma, gastric carcinoma, colon carcinoma and breast carcinoma.

10. (canceled)

11. The composition according to claim 2, wherein the plasmodium is Plasmodium vivax.

12. The composition according to claim 6, wherein the adjuvant is any one or a combination of at least two of an excipient, a diluent, a carrier, a flavoring agent, a binder and a filler.
